(19) United States
(12) Patent Application Publication
(10) Pub. No.: US 2020/0352774 A1
(43) Pub. Date: Nov. 12, 2020

(54) STABILIZATION APPARATUS AND METHODS OF USE

(71) Applicants: Daniel E. Rabinowitz, New York, NY (US); Morgan H. Rabinowitz, New York, NY (US)

(72) Inventors: Daniel E. Rabinowitz, New York, NY (US); Morgan H. Rabinowitz, New York, NY (US)

(21) Appl. No.: 16/405,658

(22) Filed: May 7, 2019

(51) Int. Cl. A61F 5/44; A61F 5/453; A61F 5/455; A61M 25/00

(52) U.S. Cl. CPC A61F 5/4408; A61F 5/453; A61M 2025/026; A61M 25/0017; A61F 5/455

(57) ABSTRACT

The present invention relates to an apparatus for attaching a medical device, preferably a urine collection device to a subject, an assembly containing the apparatus and medical device, kits containing the apparatus and methods for using the apparatus to attach, hold, secure and/or stabilize a medical device to a subject.

### FIELD OF THE INVENTION

The present invention relates to an apparatus that may be used on a variety of medical devices to attach, stabilize, hold and/or secure a medical device to a subject. More particularly, the present invention relates to an apparatus that can be used to attach, stabilize, hold and/or secure a urine collection device to a subject. The present invention also relates to an assembly that comprises the apparatus and medical device and particularly, a urine collection assembly that comprises the apparatus and a urine collection device. The present invention further relates to kits containing the apparatus, a medical device such as a urine collection device, and optional accessories that improve the use, reliability and/or comfort of the subject. The present invention additionally relates to methods for attaching, stabilizing, holding and/or securing a medical device such as a urine collection apparatus to a subject.

### DESCRIPTION OF THE RELATED ART

Catheters represent an important and commonly used medical device and the insertion of which are performed daily medical practice. Catheters are flexible tubular surgical instruments that are inserted into a body cavity to withdraw or introduce fluids. Approximately 17.5% of adult hospital inpatients in Europe and 23.6% in the US will have an indwelling urinary catheter at some point during their hospitalization. Zarb, P., et al. “The European Centre for Disease Prevention and Control (ECDC) pilot point prevalence survey of healthcare-associated infections and antimicrobial use.” EuroSurveillance Monthly 17.46 (2012): 20316, and Magill, Shelley S., et al. “Multistate point-prevalence survey of health care-associated infections.” New England Journal of Medicine 370.13 (2014): 1198-1208. In long term care facilities, up to 12% of patients across multiple nations worldwide are managed with a chronic indwelling catheter. Crnich, Christopher J., et al. “Medical device-associated infections in the long-term care setting.” Infectious Disease Clinics 26.1 (2012): 143-164; Eilers, R., et al. “Prevalence and determinants associated with healthcare-associated infections in long-term care facilities (HALT) in the Netherlands, May to June 2010.” Eurosurveillance 17.34 (2012): 20252; Moro, Maria Luisa, et al. “Infections and antimicrobial resistance in long term care facilities: a national prevalence study.” Annali di igiene: medicina preventiva e di comunita 25.2 (2013): 109-118; and Heudorf, U., K. Boehlcke, and M. Schade. “Healthcare-associated infections in long-term care facilities (HALT) in Frankfurt am Main, Germany, January to March 2011.” Eurosurveillance 17.35 (2012): 20256. A type of catheter that is inserted into the bladder is a Foley catheter.

A major problem with bladder catheters is that they often contribute to urinary tract infections due to bacteria traversing the catheter to the bladder. Another problem with bladder catheters is that over time they can become coated with a biofilm or encrusted with solids which hinder and/or obstruct drainage further contributing to the likelihood of urinary tract infections. Catheter acquired urinary tract infections (CAUTIs) are one of the most common health care acquired infections. CAUTIs may develop in as many as 75% of patients requiring a urinary catheter while in the hospital. This is especially true if the catheter is in place for several days because the risk of developing a CAUTI is between 3-7% per day. McGuckin, Maryanne. The patient survival guide: 8 simple solutions to prevent hospital-and healthcare-associated infections. Demos Medical Publishing, 2012; Lo, Evelyn, et al. “Strategies to prevent catheter-associated urinary tract infections in acute care hospitals: 2014 update.” Infection Control & Hospital Epidemiology 35.5 (2014): 464-479. Bacteriuria or candiduria is almost inevitable in approximately 50% of patients who require an indwelling Foley catheter for more than 5 days. Kunin, Calvin M. Urinary tract infections. Detection, prevention, and management. No. Ed. 5. Williams & Wilkins, 1997, pp. 227-279; Stamm, Walter E. “Catheter-associated urinary tract infections: epidemiology, pathogenesis, and prevention.” The American journal of medicine 91.3 (1991): S65-S71. Approximately 3% of patients with CAUTI bacteriuria will develop bacteremia, but given the high frequency of indwelling catheter use, it is one of the most common causes of secondary bloodstream infections in acute care facilities; all cause 30 day mortality in these CAUTI bacteremia patients can be as high as 15%. Fortin, Elise, et al. “Healthcare-Associated Bloodstream Infections Secondary to a Urinary Focus The Québec Provincial Surveillance Results.” Infection Control & Hospital Epidemiology 33.5 (2012): 456-462. In long term care facilities CAUTIs represent the source of over 50% of all episodes of bacteremia. Smith, Philip W., and Joseph M. Mylotte. “Nursing home-acquired bloodstream infection.” Infection Control & Hospital Epidemiology 26.10 (2005): 833-837; Hooton, Thomas M et al. “Diagnosis, prevention, and treatment of catheter-associated urinary tract infection in adults: 2009 International Clinical Practice Guidelines from the Infectious Diseases Society of America.” Clinical infectious diseases 50.5 (2010): 625-663. Residents with indwelling catheters in these facilities are at a 3-36 times risk of developing bacteremia compared to those patients without indwelling catheters. Nicolle, L. E. “Urinary tract infections in the elderly.” Clinics in geriatric medicine 25.3 (2009): 423-436.

Aside from the dangers associated with CAUTIs themselves, CAUTIs can lead to complications such as: prostatitis, epididymitis, and orchitis in males; and cystitis, pyelonephritis, gram-negative bacteremia, endocarditis, vertebral osteomyelitis, septic arthritis, endophthalmitis, and meningitis in all patients.

Another major problem with bladder catheters and CAUTIs is the cost of treatment. The estimated cost to treat a CAUTI ranged from approximately $876-$1,764 per patient case for non-ICU patients and up to $10,197 per case if the patient needed ICU care. Hollenbeak, Christopher S., and Amber L. Schilling. “The attributable cost of catheter-associated urinary tract infections in the United States: A systematic review.” American journal of infection control 46.7 (2018): 751-757. Patients who developed a CAUTI while inpatient had a longer length of stay as well, approximately 3.6 days and 8.1 days longer for non-ICU and ICU patients respectively. Sarah, H. Yi, et al. “Medicare reimbursement attributable to catheter-associated urinary tract infection in the inpatient setting: a retrospective cohort analysis.” Medical care 52.6 (2014): 469-478. The approximate financial burden for healthcare systems from CAUTIs is $450 million dollars per year and these costs are no longer reimbursed by insurance companies in the healthcare system. Scott, R D. The Direct Medical Costs of Healthcare-Associated Infections in U.S. Hospitals and the Benefits of Prevention. http://www.cdc.gov/ncidod/dhqp/pdf/Scott_CostPaper.pdf accessed Apr. 12, 2010. As such, there has been a push by healthcare systems as a whole to move away from the use of indwelling catheters. One way that health care providers have attempted to shift away from indwelling catheter use is by utilizing different methods of urine collection. Some of these devices include portable male and female urinals, condom catheters, clean intermittent catheterization with straight catheters, bed pans, and diapers. However, each of these devices although useful in certain circumstances all have significant downsides from both a medical and nursing care perspective.

Clean intermittent catheterization (CIC) is an excellent alternative to Foley catheter use, as it does not involve an indwelling urethral catheter, and allows for an exact urine output measurement when used. However, in order to get this output a straight catheter must be inserted through the urethra and into the bladder in order to drain the urine. The catheter is then removed and disposed. There are several problems with CIC such as it requires a catheter be inserted into a patient's urethra. This manipulation can be painful/cause discomfort, as well as a risk of infection each time a CIC is inserted (although significantly lower than the risk of having an indwelling Foley catheter). A further problem with CIC is it often requires a nurse or other medical professional trained to perform CIC to be involved in the procedure. Although a patient is able to potentially learn to do this on their own if they are a high functioning patient who will need CIC as an outpatient chronically, however, this is most often not the case for hospitalized patients or those in nursing homes. A still further problem with CIC is that there may be urine leaks or a patient may void intentionally or unintentionally in between episodes of CIC resulting in urine output that would not be collected and recorded.

One way to avoid CAUTIs and other problems associated with catheters is to avoid indwelling urine collection systems. There are a number of devices that can collect urine from outside of the body. However, each of these devices have their own shortcomings.

An example of an external urine collection device is the portable urinal. While portable urinals are commonly used in all hospitals, long-term care facilities, and other similar facilities, they come with numerous drawbacks. As an initial matter, they require the patient to perform several functions, which may be difficult and bothersome or even impossible for certain patients. For example, the patient must remember to use the urinal instead of actually getting up to go to the bathroom in order to void in the toilet as is habit. In this context, it becomes difficult or impossible to obtain accurate monitoring of a patient's urine output which is often a significant data point and indicator for many conditions. Even when a patient can remember to utilize the portable urinal, the patient must then be physically able to access and use the urinal on their own. Urinals are often kept at the bedside, or attached to the patient's bed. Patients may not be able to reach them or otherwise sit up to reach them and hold them in a manner to be able to void comfortably and without waste of the urine sample. Even if a patient is able to reach the urinal, they may be too weak, disoriented, or simply unable to use it properly, and consequently end up not voiding into the device correctly if even at all.

The above problems associated with portable urinals is especially true for patients with female genitalia attempting to use a female urinal on their own, as it is more anatomically difficult to use compared to a male urinal. In order to remedy this, often times nursing staff will need to take time away from their busy days and other patients and hold the urinal in place while the patient voids.

A further problem with portable urinals occurs in patients who have undergone certain types of procedures such as endovascular procedures with femoral access, such as a cardiac catheterization. These patients must lay completely flat for at least 4-6 hours or risk bleeding from the procedure access site. These patients are in critical need of urine output monitoring, but Foley catheters are often not used due to their risk of infection. In this case often times a portable urinal is used for monitoring, however, the patient is rarely able to perform using the portable urinal on his or her own. The use of the portable urinal is completely up to the nursing staff, and it can be a frequent act for them to perform, taking them away from many other more pressing duties.

A still further problem with portable urinal use is they may spill over, and if the output was not recorded in time the urine output data is lost.

Condom catheters also known as “Texas catheters” have been frequently used as external catheters in men but they are unable to be used by women. The condom catheter is essentially a condom that is rolled onto a penis and secured with tape. The condom catheter is equipped with a drainage component, i.e., a bag or similar collection device, so that when a patient voids, the urine is collected and recorded. A major problem with the use of condom catheters is that they are very insecure. It is difficult to roll the condoms onto a flaccid penis and have them remain in place without falling off. Further, the inside of the condom becomes wet/moist and allows for skin breakdown and is an ideal environment for infection.

Bedpans are commonly used for women in the hospital who need to void, but there are many difficulties with them. The first issue is that it often requires significant nursing staff to help place the bed pan underneath a patient as these patients often time must be rolled or lifted. Patients typically do not have access to bed pans without a nurse, so they may have to wait for a nurse to obtain a bed pan and bring it to them. This further occupies a nurse's time and often time patients cannot wait and will void in the bed. The bed pan is also very uncomfortable for patients and the positioning of the patients does not facilitate easy voiding. There is also a risk of urine spillage with a bedpan resulting in inappropriately measured urine.

Diapers are often used for incontinent patients, however, there are many problems associated with them as well. The first of which is the measurement of urine is not precise, so outputs cannot be measured well. More importantly, patients often end up sitting in their own wet diapers before it is changed. This results in discomfort for patients and the potential for skin break down and infection. Finally, there is also a nursing work requirement that they must monitor the patients' diapers and change and replace them when needed which takes up even more valuable nursing time.

Hence, a need exists in the art to provide a way to attach, stabilize, hold and/or secure a medical device such as a portable urinal to a subject. In particular, a need still exist in the art to provide a way to attach, stabilize, hold and/or secure a urine collection device to a subject that will decrease the frequency of CAUTIs and is convenient and simple to use.

Accordingly, it is an object of the present invention to provide an apparatus that attaches, stabilizes, holds and/or secures medical devices to a subject.

It is an additional object of the present invention to provide an apparatus that attaches, stabilizes, holds and/or secures medical devices such as portable urinals to a subject that is secure, reliable, convenient, comfortable and simple to use.

It is also an object of the present invention to provide a urine collection assembly comprising a portable urinal and an apparatus that attaches, stabilizes, holds and/or secures the portable urinal to a subject.

It is a further object of the present invention to provide improved means to effectively and comfortably collect urine from a subject.

### SUMMARY OF THE INVENTION

The above objectives and others are obtained by the present invention, which is a an apparatus that attaches, stabilizes, holds and/or secures medical devices to a subject wherein the apparatus comprises a means for attaching the apparatus to the medical device and a means for attaching the apparatus to the subject. In certain embodiments the medical device is a portable urinal or an external catheter.

In certain embodiments the means for attaching the apparatus to the medical device comprises a strap, strip, tape, band, belt or ribbon of material such as cloth, plastic, leather or combinations thereof that may be attached or secured to the outer surface of the medical device. The strap, strip, tape, band, belt or ribbon of material may be attached or secured to the outer surface of the medical device by the use of fastening means such as hooks, clips, snaps, hook and eye material (VELCRO), elastic material, adhesive material such as glue or adhesive tape or combinations of the forgoing. In certain preferred embodiments, the means for attaching the apparatus to the medical device comprises a strap, strip, tape, band, belt or ribbon of material with one or more collars, loops or rings that engage or surround the outer surface of the medical device and secures the apparatus to the medical device.

In certain embodiments the means for attaching the apparatus to the subject comprises a strap, strip, tape, band, belt or ribbon of material such as cloth, plastic, leather or combinations thereof that may be attached or secured to the subject. In embodiments wherein the medical device is a portable urinal or external catheter, the means for attaching the apparatus to the subject will engage or attach to one or both legs of the subject, engage or attach to the hip or waist region of the subject or a combination of one or both legs of the subject and the hip or waist region of the subject. The strap, strip, tape, band, belt or ribbon of material may be engaged or attached to the subject by wrapping it around a portion of the subject such as a leg or waist and securing it by the use of fastening means such as buckles, buttons, safety pins, hooks, clips, snaps, hook and eye material (VELCRO), elastic material, adhesive material such as glue or adhesive tape or combinations of the forgoing.

Embodiments of the present invention provide an efficient, comfortable and convenient means to collect urine. These embodiments also prevent spillage, or waste of urine and provide an improved method for collecting, measuring and/or studying urine.

It is another object of the present invention to provide an apparatus which is sufficiently versatile to be used on different types and styles of existing portable urinals or external catheters without the need for substantial modifications or multiple embodiments of the apparatus. Preferably the apparatus of the present invention can be used on all types of commercially available male and female portable urinals as well as external catheters and does not need multiple or customized embodiments of the apparatus to accommodate use with specific types of male and female portable urinals as well as external catheters.

It is an object of the present invention to provide a urine collection assembly that comprises a urine collection device and an apparatus that attaches, stabilizes, holds and/or secures the urine collection device to a subject. The present invention further relates to method of using the urine collection assembly to collect, measure and/or study urine from a subject.

The present invention further relates to kits containing a urine collection device, an apparatus that attaches, stabilizes, holds and/or secures the urine collection device to a subject, optionally printed instructions for using the urine collection device and apparatus and optionally additional attachment accessories for the improved collection and/or comfort of the subject. The present invention further relates to method of using the kits to collect, measure and/or study urine from a subject.

### BRIEF DESCRIPTION OF THE DRAWINGS

FIG. 1a is a top perspective view of one embodiment of the apparatus of the present invention;

FIG. 1b is a top perspective view of the one embodiment of the apparatus of the present invention;

FIG. 2a is a top perspective view of one embodiment of a urine collection assembly of the present invention;

FIG. 2b is a top perspective view of one embodiment of a urine collection assembly of the present invention;

FIG. 3a is a top perspective view of one embodiment of a urine collection assembly of the present invention;

FIG. 3b is a top perspective view of one embodiment of a urine collection assembly of the present invention;

FIG. 4 is a top plan view of the urine collection assembly of the present invention on a human subject;

FIG. 5a is a top perspective view of an embodiment of the present invention with an optional drainage feature;

FIG. 5b is a top perspective view of an embodiment of the present invention with the use of a drain member and valve member of the optional drainage feature;

FIG. 6a is a top plan view of an embodiment of the fastening means of the apparatus of the present invention in a non-engage mode;

FIG. 6b is top plan view of an embodiment of the fastening means of the apparatus of the present invention in an engaged mode;

FIG. 7a is a top plan view of an embodiment of the fastening means of the apparatus of the present invention in a non-engaged mode;

FIG. 7b is a top plan view of an embodiment of the fastening means of the apparatus of the present invention in an engaged mode;

FIG. 8a is a top plan view of an embodiment of the fastening means of the apparatus of the present invention in a non-engaged mode;

FIG. 8b is a top plan view of an embodiment of the fastening means of the apparatus of the present invention in an engaged mode;

FIG. 9a is a top perspective view of one embodiment of the apparatus of the present invention;

FIG. 9b is a top perspective view of one embodiment of the apparatus of the present invention;

FIG. 10a is a top perspective view of one embodiment of the urine collection assembly of the present invention; and

FIG. 10b is a top perspective view of one embodiment of the urine collection assembly of the present invention.

### DETAILED DESCRIPTION OF THE INVENTION

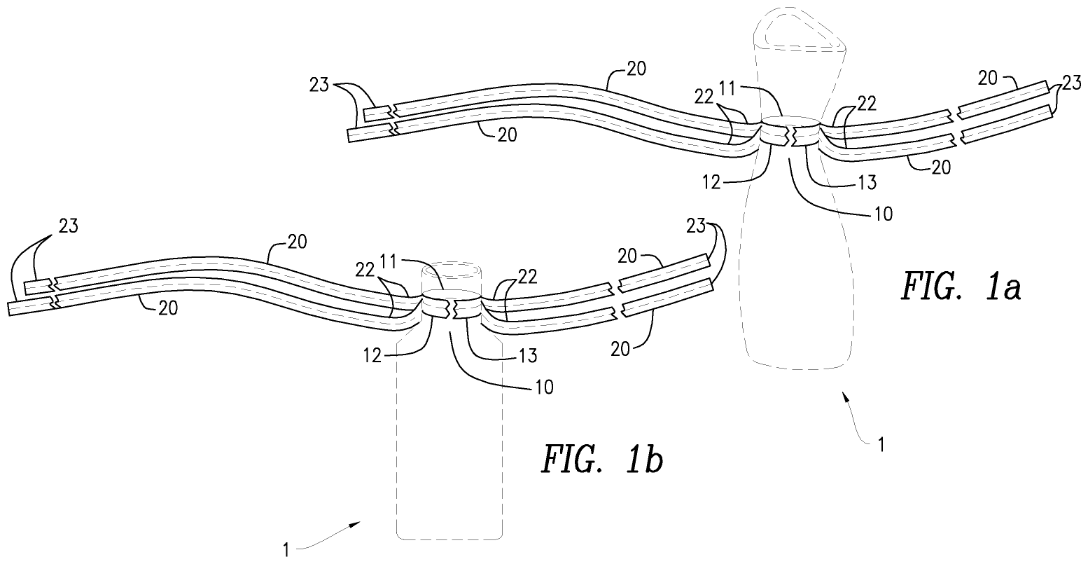

The following is a detailed description of exemplary embodiments of the present invention with reference to FIGS. 1-10. FIGS. 1-10 are for illustration purposes only and are not to scale.

As used herein the meaning of “a”, “an”, and “the” includes plural reference unless the context clearly dictates otherwise. Also, as used herein the meaning of “in” includes “in” and “on” unless the context clearly dictates otherwise.

As used herein the term “subject” includes mammals such as humans, monkeys, cows, horses, dogs, cats, pigs etc. and preferably a human subject.

The present invention relates to an apparatus, which can be used to attach, stabilize, hold and/or secure a medical device in relation to a subject's body, subject wherein the apparatus comprises: (i) a means for attaching the apparatus to the medical device and (ii) a means for attaching the apparatus to the subject. In certain embodiments the medical device is a urine collection device such as a portable urinal or an external catheter. The present invention also includes assemblies that comprise the medical device, preferably a portable urinal or external catheter, and the apparatus and use of the assembly, preferably to collect urine from a subject. The present invention further includes a kit comprising: (i) the medical device, preferably a urine collection device such as a portable urinal or external catheter; (ii) the apparatus; optionally (iii) printed instructions for using the medical device and apparatus to attach, stabilize, hold and/or secure the medical device to a subject assembly, preferably to collect urine from a subject and optionally (iv) additional accessories to aid in the comfort and effective use of the medical device and assembly. An alternative embodiment of the kit comprises: (i) the apparatus; optionally (ii) printed instructions for using the apparatus with a medical device, preferably a urine collection device such as a portable urinal or external catheter; and optionally (iii) additional accessories to aid in the comfort and effective use of the medical device and assembly.

The means for attaching the apparatus to the medical device (i) comprises at least on strap, strip, tape, band, belt or ribbon of material (hereinafter collectively referred to as “strap” unless otherwise indicated). The strap (i) may be made of a woven or non-woven fabric, plastic, leather or combinations thereof. The woven or non-woven fabric may be made of natural materials such as cotton or wool, synthetic materials such as nylon or polyester or a combination of natural and synthetic materials. The strap (i) may be elastic or non-elastic. The strap (i) may be attached to the outer surface of the medical device by the use of fastening means such as hooks, clips, snaps, hook and eye material (VELCRO), elastic material, adhesive material such as glue or adhesive tape or combinations of the forgoing.

The means for attaching the apparatus to the subject (ii) comprises at least one strap. The strap (ii) may be made of a woven or non-woven fabric, plastic, leather or combinations thereof. The woven or non-woven fabric may be made of natural materials such as cotton or wool, synthetic materials such as nylon or polyester or a combination of natural and synthetic materials. The strap (ii) may be elastic or non-elastic. In embodiments wherein the medical device is a portable urinal or external catheter, the strap (ii) will engage or attach to one or both legs of the subject, engage or attach to the hip or waist region of the subject or a combination of one or both legs of the subject and the hip or waist region of the subject. The strap (ii) may be engaged or attached to the subject by wrapping it around a portion of the subject such as a leg or waist and securing it by the use of fastening means such as buckles, buttons, safety pins, hooks, clips, snaps, hook and eye material (VELCRO), elastic material, adhesive material such as glue or adhesive tape or combinations of the forgoing.

The strap (i) and strap (ii) may be a single unitary structure. The unitary structure may be formed by permanently attaching strap (i) to strap (ii). The permanent attaching can be accomplish by known methods such as stitching, sewing, weaving, adhering or combinations thereof. Alternatively, the unitary structure may be formed by releasably attaching strap (i) and strap (ii) by known methods such as buckles, buttons, safety pins, hooks, clips, snaps, hook and eye material (VELCRO) or combinations thereof.

In certain embodiments the apparatus comprises: i) an adjustable collar, loop or ring (hereinafter referred to a “collar member”), wherein the collar member is a means for attaching the apparatus the outer surface of the medical device; and ii) at least one adjustable strap member wherein the at least one adjustable strap member is a means for attaching the medical device to a subject's body.

More specifically, the apparatus 1 shown in FIGS. 1a, 1b, 2a, 2b, 3a, 3b, 4, 9a, 9b, 10a, and 10b includes an adjustable collar member 10, and at least one adjustable strap member 20.

The collar member 10 is a means for attaching, stabilizing, holding and/or securing the apparatus 1 to a medical device wherein the collar member 10 can be adjusted to encircle or surround a portion of the outer surface of the medical device. In one embodiment, the collar member 10 is self-adjusting such as by the use of an elastic material to form the collar member 10. In another embodiment, the collar member 10 is manually adjustable such as by the use of fastening means as shown in FIGS. 6a, 6b, 7a, 7b, 8a and 8b. In one embodiment, the collar member 10 encircles a portion of the medical device by manually placing the medical device through the center opening 11 of the collar member 10. In another embodiment, the collar member 10 is comprised of a first collar-strap end 12 and a second collar-strap end 13 that encircles a portion of the medical device by fastening means.

The collar member 10 may be formed as a unitary structure in a circular shape with a central opening 11 by molding, casting, machining, or by any known method to create the desired structure. The collar member 10 may also be formed as a unitary structure in an elongated strap shape by molding, casting, machining, or any known method to create the desired structure. In this aspect of the embodiment, the collar member is an elongated strap composed of a first collar-strap end 12 that is attached or attachable to a second collar-strap end 13 by fastening means.

The collar member 10 can be made of any type of flexible material. Suitable materials include, for example, natural materials such as cloth, rope, leather and combinations thereof; synthetic materials such as polymeric materials including plastic, rubber, elastic, latex, silicone, nylon, polyester, polypropylene, vinyls and combinations thereof and combinations of natural and synthetic materials. The collar member 10 may be any suitable length, width, thickness and length. In a preferred embodiment, the collar member 10 is formed of an elastic material.

The apparatus 1 further includes at least one adjustable strap member 20. The at least one adjustable strap member 20 is a means for attaching, stabilizing, holding and/or securing the apparatus 1 to a subject's body. In one embodiment, the one strap member 20 is self-adjusting such as by the use of an elastic material to form the strap member 20. In another embodiment, the strap member 20 is manually adjustable such as by the use of fastening means as shown in FIGS. 6a, 6b, 7a, 7b, 8a and 8b. In one embodiment, the strap member 20 encircles a portion of the subject's body by manually placing the portion of the subject's body through the center opening 21 of the strap member 20. In another embodiment, the strap member 20 is comprised of a first strap end 22 and a second strap end 23 that encircles a portion of a subject's body by fastening means.

The strap member 20 may be formed as a unitary structure in a circular shape with a central opening 21 by molding, casting, machining, or by any known method to create the desired structure. The strap member 20 may also be formed as a unitary structure in an elongated strap shape by molding, casting, machining, or any known method to create the desired structure. In this aspect of the embodiment, the strap member 20 is an elongated strap composed of a first strap end 22 that is attached or attachable to a second strap end 23 by fastening means.

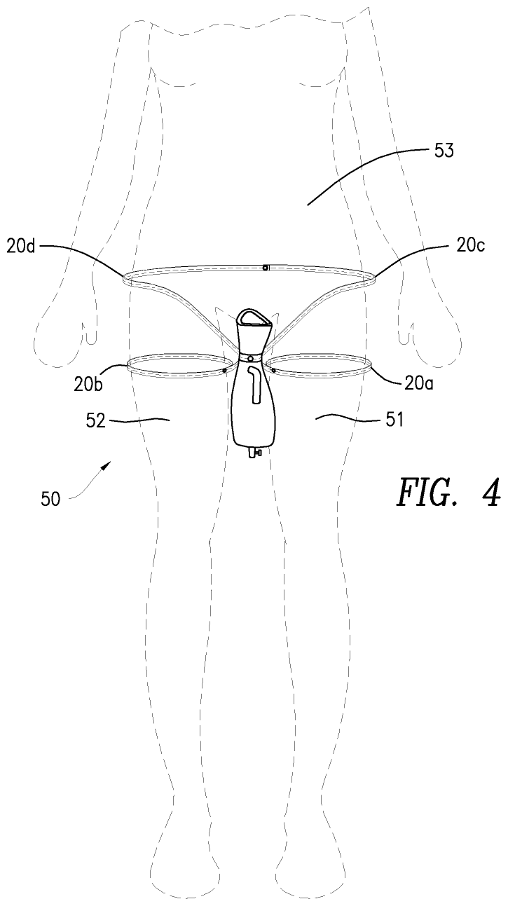

Further, strap member 20 is attached or attachable to the collar member 10 in any configuration suitable to comfortably and effectively attach, stabilize, hold and/or secure the apparatus 1 to a subject's body 50 and the medical device against a subject's body 50 as shown in FIG. 4. In a preferred embodiment, the apparatus 1 of the present invention includes more than one strap member 20, each strap member 20 being attached or attachable to the collar member 10 and/or to any other strap member 20, or multiple strap members thereof, in any configuration suitable to comfortably and effectively secure apparatus 1 to a subject's body 50 and the medical device against a subject's body 50.

In one embodiment the apparatus 1 of the present invention comprises at least two strap members, 20a and 20b, wherein the first strap member 20a and second strap member 20b are attached or attachable to collar member 10 by any suitable means, preferably by fastening means, at any suitable location along collar member 10, preferably on opposite sides of collar member 10. In a preferred embodiment, first strap member 20a is attached or attachable to collar member 10, wherein first strap end 22a is attached or attachable to collar member 10 preferably by fastening means and second strap end 23a is attached or attachable to first strap end 22a or collar member 10, preferably by fastening means. In this aspect of the embodiment, second strap member 20b is attached or attachable to collar member 10, preferably by fastening means, at any suitable location along collar member 10. In a preferred embodiment, first strap member 20b is attached or attachable to collar member 10, wherein first strap end 22b is attached or attachable to collar member 10, preferably by fastening means, and second strap end 23b is attached or attachable to first strap end 22b or collar member 10, preferably by fastening means.

In another embodiment the apparatus 1 of the present invention comprises at least three or four strap members, wherein a third strap member 20c and a fourth strap member 20d are attached or attachable to collar member 10 by any suitable means, preferably by fastening means, at any suitable location along collar member 10, preferably on opposite sides of collar member 10, and wherein the third strap member 20c and the fourth strap member 20d are further attached or attachable to each other, preferably by fastening means, at any suitable location along the opposite strap members. In a preferred embodiment, the third strap member 20c is attached or attachable to collar member 10 and the fourth strap member 20d, wherein a first strap end 22c is attached or attachable to collar member 10, preferably by a fastening means, and second strap end 23c is attached or attachable to the fourth strap member 20d. In this aspect of the invention, fourth strap member 20d is attached or attachable to collar member 10 and the first strap member 20c, wherein, a first strap end 22d is attached or attachable to collar member 10, preferably by a fastening means, a second strap end 23d is attached or attachable to the third strap member 20c.

In another embodiment, apparatus 1 comprises at least four strap members, 20a, 20b, 20c, and 20d, wherein first strap member 20a, second strap member 20b, third strap member 20c and fourth strap member 20d are attached or attachable to collar member 10 by any suitable means, preferably by a fastening means, at any suitable location along collar member 10, wherein third strap member 20c and fourth strap member 20d are further attached or attachable to each other. In this embodiment, first strap member 20a is attached or attachable to the collar member 10 by first strap end 22a preferably by fastening means, and second strap end 23a is attached or attachable to the collar member 10 or first strap end 22a, preferably by fastening means. Second strap member 20b is attached or attachable to the collar member 10 by first strap end 22b preferably by fastening means, and second strap end 23b is attached or attachable to the collar member 10 or first strap end 22b, preferably by fastening means. Third strap member 20c is attached or attachable to the collar member 10 by a first strap end 22c, preferably by fastening means, and to fourth strap member 20d by a second strap end 23c, preferably by fastening means; fourth strap member 20d is also attached or attachable to the collar member 10 by a first strap end 22d, preferably by fastening means, and to third strap member 20c by a second strap end 23d, preferably by fastening means.

The at least one strap member 20, may be in any configuration suitable to comfortably and effectively secure apparatus 1 to a subject's body 50 and the medical device against a subject's body 50. In a preferred embodiment, the apparatus 1 of the present invention includes more than one strap member 20, each strap member 20 being attached or attachable to the collar member 10 and/or to any other strap member 20, or multiple strap members thereof, in any configuration suitable to comfortably and effectively secure apparatus 1 and the medical device to a subject's body 50.

For example, strap member 20a may secure the left leg, left-side groin, or the left-side hip of a subject's body. In another example, strap member 20b may secure the right leg, right-side groin, or the right-side hip of a subject's body 50. In another example, strap member 20c and 20d may secure the waist, hips, buttocks, or upper thighs of a subject's body 50.

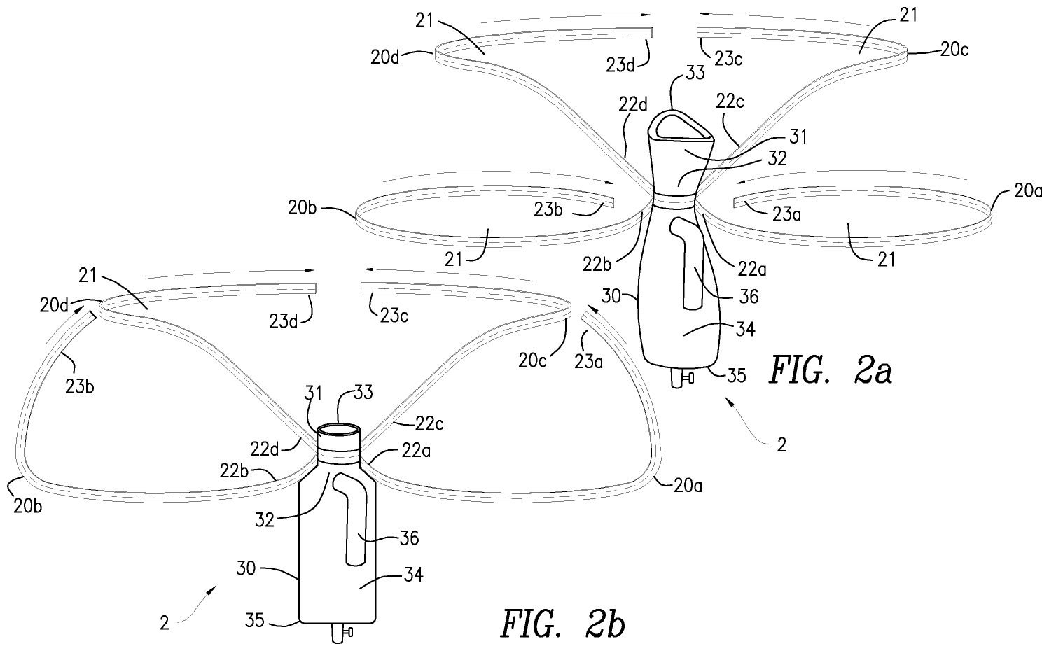

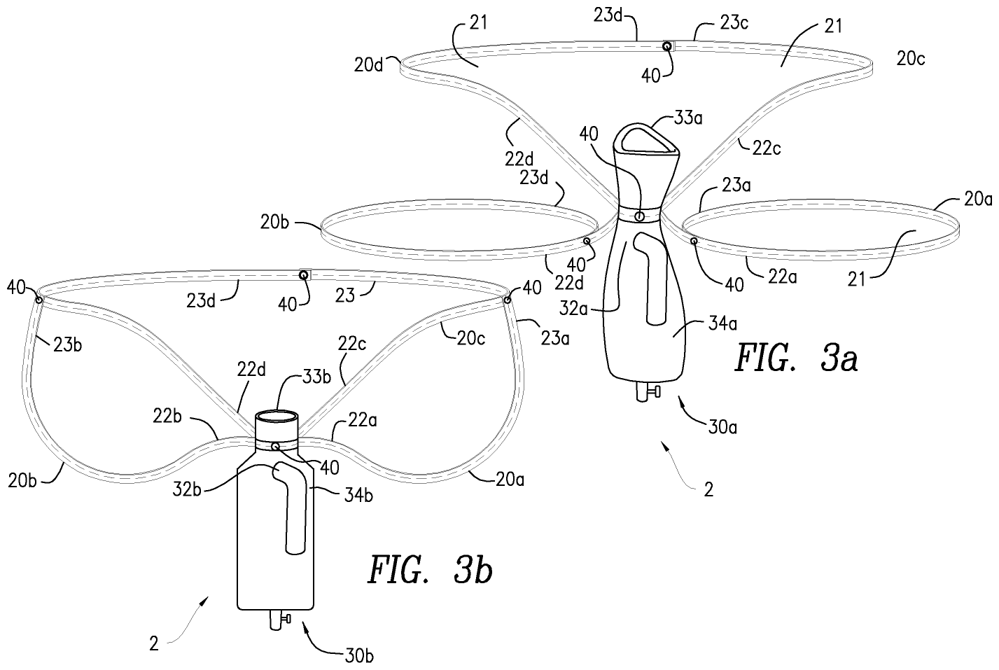

As seen in FIGS. 2a and 3a, in one embodiment strap member 20a may secure the left leg of subject's body, strap member 20b may secure the right leg of a subject's body, and strap members 20c and 20d may secure the waist 53 of a subject's body.

As seen in FIGS. 2b and 3b, in one embodiment strap member 20a may secure the left side groin of subject's body, strap member 20b may secure the right side groin of a subject's body, and strap members 20c and 20d may secure the waist 53 of a subject's body.

In a preferred embodiment, as shown in FIG. 4, strap member 20a may secure the left leg 51 of a subject's body, strap member 20b may secure the right leg 52 of a subject's body, and strap members 20c and 20d may secure the waist 53 of a subject's body.

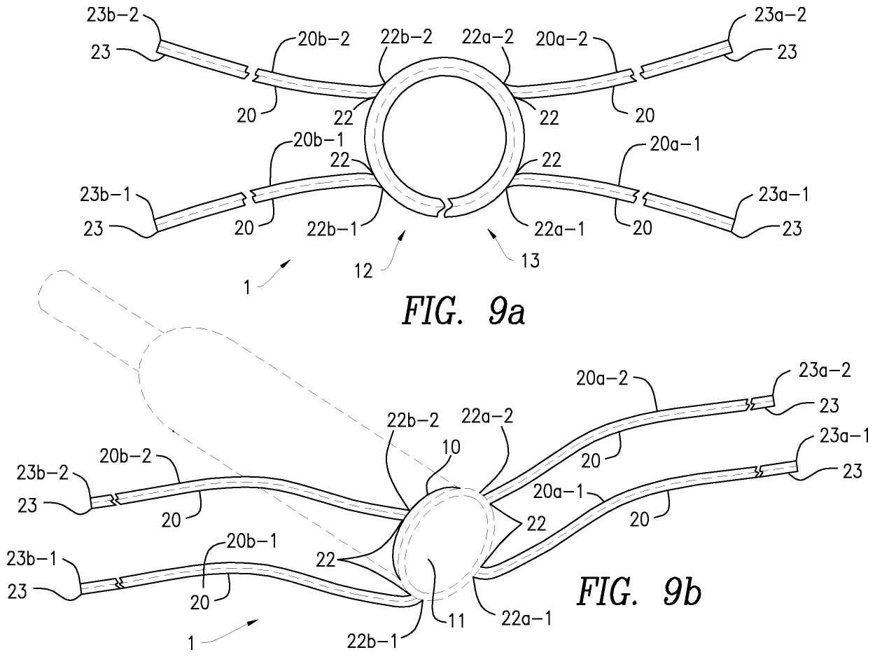

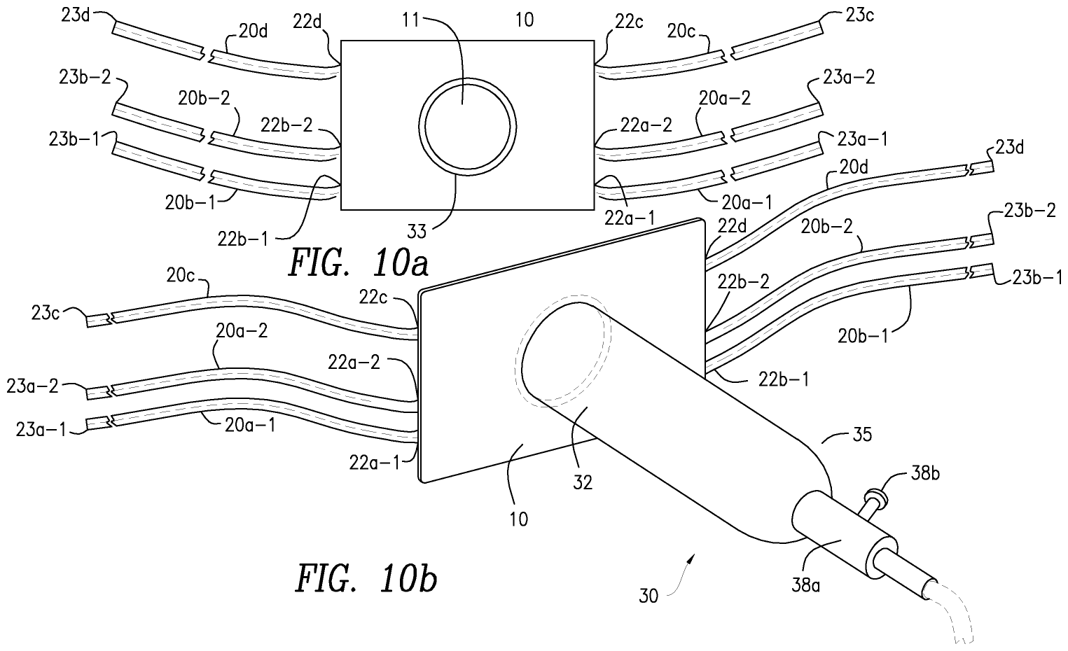

According to some embodiments, the apparatus 1 of the present invention includes more than one strap member 20a-1, 20a-2, 20b-1 and 20b-2 wherein strap member 20a-1 may secure the anterior side of the left leg of subject's body, strap member 20a-2 may secure the posterior side of the left leg of subject's body, 20b-1 may secure the anterior side of the right leg of subject's body, strap member 20b-2 may secure the posterior side of the right leg of subject's body (FIGS. 9a, 9b, 10a, 10b). In another embodiment, the stabilization device of the present invention includes more than one strap member 20a-1, 20a-2, 20b-1 and 20b-2, 20c, and 20d wherein strap member 20a-1 may secure the anterior side of the left leg of subject's body, strap member 20a-2 may secure the posterior side of the left leg of subject's body, 20b-1 may secure the anterior side of the right leg of subject's body, strap member 20b-2 may secure the posterior side of the right leg of subject's body, and strap members 20c and 20d may secure the waist 53 of a subject's body (FIGS. 10a and 10b).

The at least one strap member can made of any type of material as previously described and are provided in suitable length, width, and thickness to accommodate various subject dimensions. In a preferred embodiment, the strap member is adjustable.

A fastening means 40 can be formed by any technique known in the art such as machining, and can be formed during the manufacturing process of the apparatus 1 or may be formed as a separate part and later affixed to the apparatus 1. The fastening means 40 may separably-fasten or may not separably fasten. A fastening means 40 that does not separably-fasten may be formed by any suitable means including, but not limited to, stitching, adhering with a suitable adhesive, or heat bonding. Fastening means 40 that may separably-fasten can be made of any type of material. Suitable materials include, but are not limited to, natural materials such as metal, wood, and cloth; chemical materials such as adhesives; and polymer materials such as, plastic, rubber, latex, silicone, nylon, polyester, and vinyl. Separably-fastening means can be any type of complementary fastening system suitable including but not limited to, a slide system, buckle system, clasp system, pin system, drawstring system, flaccid system, and a hook and loop system.

A buckle system allows a first collar-strap end 12 or a first strap end 22 affixed with a male buckle member to separably-fasten to a complementary female buckle member affixed to an opening member in a second collar-strap end 13 or a second strap end 23 by allowing the insertion of the male buckle member into the female buckle member or into the opening member in a manner that prevents or hinders separation thereof. In one embodiment, the collar member or the strap member is comprised of more than one female buckle member or opening member. In another embodiment, the buckle system may have an additional means to allow the length of the collar member or the strap member to be adjusted. In a preferred embodiment, the buckle system is adjustable. Types of buckle systems include, but are not limited to prong buckles, tongue buckles, and side-release buckles.

A pin system allows a first collar-strap end 12 or a first strap end 22 affixed with a male pin member to separably-fasten to a female pin member or an opening in a second collar-strap end 13 or a second strap end 23 by allowing the male pin member to impale or penetrate into the female pin member or the opening member in a manner that prevents or hinders separation thereof. In one embodiment, the collar member is comprised of more than one opening or female pin members. In one embodiment, the pin system is adjustable. In another embodiment, the pin system may have an additional means to allow the length of the collar member or the strap member to be adjusted. Types of pin systems include, but are not limited to, buttons, snap fasteners, clevis pins, T-pins, and cotter pins.

In a preferred embodiment, the fastening means is a pin system. As shown in FIGS. 6a and 6b, in a preferred embodiment, the collar member or strap member comprises a male pin member 41 and a female pin member or opening member 42.

A clasp system allows a first collar-strap end 12 or a first strap end 22 affixed with a male clasp member to separably-fasten to an affixed complementary female clasp member, opening member, or to the material of a second collar-strap end 13 or a second strap end 23 by allowing the male clasp member to engage with the female clasp member or opening member in a jaw-like manner that prevents or hinders separation thereof. In one embodiment, there are more than one male clasp members, female clasp members, or openings thereof. In one embodiment, the clasp system is adjustable. In another embodiment, the clasp system may have an additional means to allow the length of the collar member or the strap member to be adjusted. Types of clasp systems include, but are not limited to, hook clasps.

A hook and loop system allows a first collar-strap end 12 or a first strap end 22 affixed with a hook member to separably fasten a second collar-strap end 13 or a second strap end 23 affixed with a loop member, wherein the hook member and the loop member are on opposing faces of their respective collar-strap end or strap end, and by allowing the hook member and loop member to engage in an anti-parallel manner that prevents or hinders separation thereof. In one embodiment, the hook and loop system is adjustable. In another embodiment, the hook and loop system may have an additional means to allow the length of the collar member or the strap member to be adjusted. Types of hook and loop systems include, but are not limited to, VELCRO.

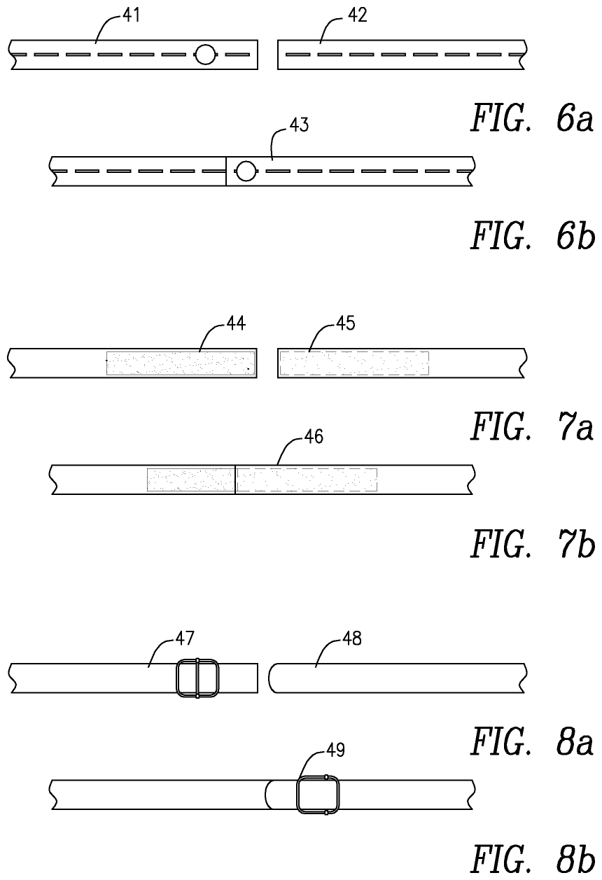

In a preferred embodiment, the fastening means is a hook and loop system. As shown in FIGS. 7a and 7b, in a preferred embodiment, the collar member or strap member comprises a hook member 44 and a loop member 45 to become separably-fastened 46.

A drawstring system allows a first collar-strap end 12 or a first strap end 22 to separably-fasten to a second collar-strap end 13 or a second strap end 23 by allowing collar-strap end and a strap end pass through a toggle member wherein the toggle member prevents or hinders the separation thereof. In one embodiment, the drawstring system is adjustable. In another embodiment, the drawstring system may have an additional means to allow the length of the collar member or the strap member to be adjusted. Types of drawstring systems include, but are not limited to, cord locks, cord fasteners, cord toggles, cord stoppers, and spring toggles.

A slide system allows a first collar-strap end 12 or a first strap end 22 to separably-fasten to a second collar-strap end 13 or a second strap end 23 by allowing a first collar-strap end 12 or first strap end 22 to pass through an aperture in an affixed female slide member on a second collar-strap end 13 or a second strap end 23, in a path generally anti-parallel, wherein the slide member is designed to hinder or prevent separation thereof. In one embodiment, the slide system is adjustable. In another embodiment, the slide system may have an additional means to allow the length of the collar member or the strap member to be adjusted. Types of slide systems include, but are not limited to, buckle trims, and buckle slides.

In a preferred embodiment, the fastening means is a slide system. As shown in FIGS. 8a and 8b, in a preferred embodiment, the collar member or strap member comprises a female slide member 47, a collar-strap end or strap-end 48 to become separably-fastened 49.

A flaccid system allows a first collar-strap end 12 or a first strap end 22 to separably fasten with a second collar-strap end 13 or a second strap end 23 by allowing collar-strap ends and/or the strap ends to be frictionally secured in a manner that prevents or hinders the separation thereof. In one embodiment, the flaccid system is adjustable. In another embodiment, the flaccid system may have an additional means to allow the length of the collar member or the strap member to be adjusted. Types of flaccid systems include, but are not limited to, lacing or tying.

Additional means to adjust the length of the collar member or strap member may be any means suitable to shorten the length of the collar member or strap member conveniently, simply, and quickly.

In a preferred embodiment, the medical device is a urine collection device. In one embodiment, urine collection device is a portable male or female urinal or an external catheter. As shown in FIGS. 2a, 2b, 3a and 3b, the urine collection device is a portable urinal 30. As shown in FIGS. 9a, 9b, 10a and 10b, the urine collection device is an external catheter 30.

In this aspect of the invention, the collar member 10 is a means for attaching, stabilizing, holding and/or securing the apparatus 1 to a portable urinal 30 wherein the collar member 10 can be adjustably secured to encircle a portion of the portable urinal 30 and the at least one strap member 20 is a means for securing the apparatus 1 and the portable urinal 30 to a portion of a the body of a subject 50 wherein the at least one strap member 20 can be adjustably secured to encircle a portion of the subject's body 50.

In one embodiment, the urinal 30 is attached to the apparatus 1 wherein the collar member 10 is self-adjusting. In another embodiment, the urinal 30 is secured in the apparatus 1 wherein the collar member 10 is manually adjustable. In one embodiment, the collar member 10 encircles a portion of the urinal 30 by manually placing the head 31 of the urinal 30 through the center opening 11 of the collar member 10. In another embodiment, the collar member 10 is comprised of a first collar-strap end 12 and a second collar-strap end 13 that encircles a portion of urinal 30 by fastening means.

The at least one strap member 20, may be in any configuration suitable to comfortably and effectively secure the apparatus 1 to a subject's body 50 and the portable urinal against a subject's body 50. In one embodiment, the apparatus 1 of the present invention includes more than one strap member 20a, 20b, 20c, and 20d wherein strap member 20a may secure the left leg of subject's body, strap member 20b may secure the right leg of a subject's body, and strap members 20c and 20d may secure the waist 53 of a subject's body (FIGS. 2a, 3a, and 4). In another embodiment, the apparatus 1 of the present invention includes more than one strap member 20a, 20b, 20c, and 20d wherein strap member 20a may secure the left side groin of subject's body, strap member 20b may secure the right side groin of a subject's body, and strap members 20c and 20d may secure the waist 53 of a subject's body (FIGS. 2b, and 3b).

According to some embodiments of the present invention, the urine collection 30 device is an external catheter, referred to sometimes herein as condom catheter or condom. In this aspect of the invention, the collar member 10 is a means for securing the stabilization device 1 to the external catheter 30 wherein the collar member 10 can be adjustably secured to encircle the opening of the external catheter 30 and the at least one strap member 20 is a means for securing the apparatus 1 and the external catheter 30 to a portion of the body of a subject 50 wherein the at least one strap member 20 can be adjustably secured to encircle a portion of the subject's body 50.

In one embodiment, the external catheter 30 is secured in the stabilization device 1 wherein the collar member 10 is self-adjusting. In another embodiment, the external catheter 30 is secured in the apparatus 1 wherein the collar member 10 is manually adjustable. In one embodiment, the collar member 10 encircles a portion of the external catheter 30 by manually fitting the open portion of the condom through the center opening 11 of the collar member 10. In another embodiment, the collar member 10 is comprised of a first collar-strap end 12 and a second collar-strap end 13 that encircles the base of the external catheter 30 by fastening means.

In this aspect of the invention, the collar member 10 is a means for securing the apparatus 1 to the external catheter 30 wherein the collar member 10 can be adjustably secured to encircle a portion of the external catheter 30 and the at least one strap member 20 is a means for securing the apparatus 1 and the external catheter 30 to a portion of a the body of a subject 50 wherein the at least one strap member 20 can be adjustably secured to encircle a portion of the subject's body 50.

The at least one strap member 20, may be in any configuration suitable to comfortably and effectively attach, stabilize hold and/or secure the apparatus 1 to a subject's body 50 and the external catheter 30 against a subject's body 50. In one embodiment, the apparatus 1 of the present invention includes more than one strap member 20a-1, 20a-2, 20b-1 and 20b-2 wherein strap member 20a-1 may secure the anterior side of the left leg of subject's body, strap member 20a-2 may secure the posterior side of the left leg of subject's body, 20b-1 may secure the anterior side of the right leg of subject's body, strap member 20b-2 may secure the posterior side of the right leg of subject's body (FIGS. 9a, 9b, 10a, 10b). In another embodiment, the apparatus 1 of the present invention includes more than one strap member 20a-1, 20a-2, 20b-1 and 20b-2, 20c, and 20d wherein strap member 20a-1 may secure the anterior side of the left leg of subject's body, strap member 20a-2 may secure the posterior side of the left leg of subject's body, 20b-1 may secure the anterior side of the right leg of subject's body, strap member 20b-2 may secure the posterior side of the right leg of subject's body, and strap members 20c and 20d may secure the waist 53 of a subject's body (FIGS. 10a and 10b).

The urine collection device may be any container suitable to collect urine. In one embodiment, urine collection device 30 is hollow inside and includes a body portion 34, a bottom portion 35, a neck portion 32, a head portion 31, and a mouth portion 33 that is an opening to collect urine, where the body portion 34 is a shape that is any shape suitable to collect urine, preferably essentially square or circular in cross section, the bottom portion is a shape that is suitable to collect urine, the neck portion 32 is any shape suitable to contain urine, and the mouth portion 33 is any shape suitable to be fitted to a subject's body and collect urine comfortably.

As described above the urine collection device may be a portable urinal. As seen in FIGS. 2a and 2b, the urine collection device includes existing portable urinals that are currently commercially available. The portable urinals may shaped for subjects with female presenting genitalia or for subjects with male presenting genitalia. For the purposes of this application, the male and female urinal will be referred to as “urinal,” unless otherwise indicated.

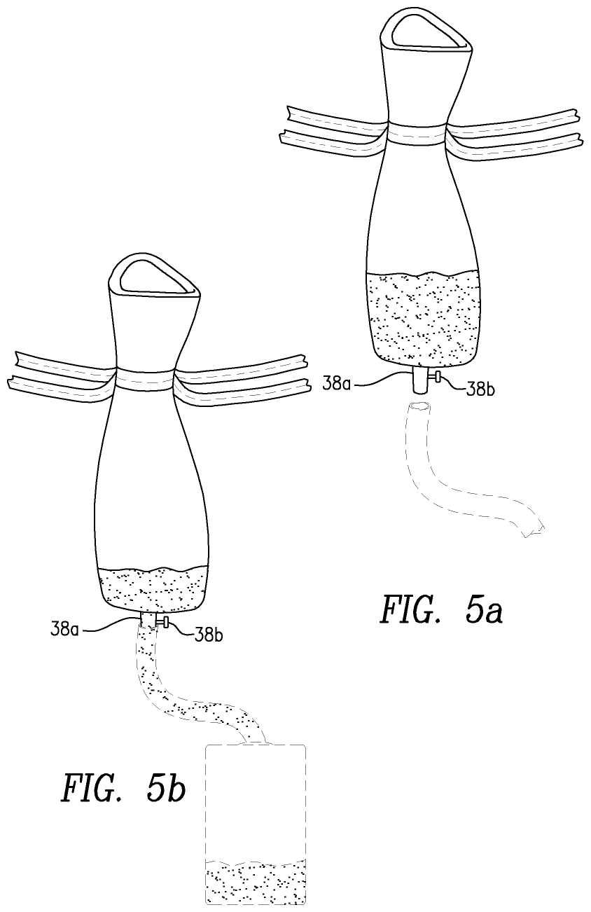

As seen in FIGS. 3a, 3b, 5a and 5b, the urine collection device may also utilize a portable urinal that employs a collection facilitating devices such as drain member 38 described in greater detail below.

In one embodiment, the urine collection device 30 is hollow inside and includes a body portion 34 that collects urine, a bottom portion 35, a neck portion 32, a head portion 31, and a mouth portion 33 that is an opening to collect urine, where the body portion 34 is a shape that is any shape suitable to collect urine, preferably essentially square or circular in cross section, the bottom portion is a shape that is suitable to collect urine, the neck portion 32 extends angular away from the body portion 34 and is a shape that is essentially narrower or pinched in comparison to the body portion 34 and the mouth portion 33, and where the mouth portion 33 is a shape that allows to be fitted to a subject's body and collect urine comfortably.

The urinal of the present invention can be made of any type of suitable material, preferably any suitable plastic, including, polyethylene.

In one embodiment, the urine collection device of the present invention is a shape that is suitable for a subject with female presenting genitalia. In this aspect of the invention, the urinal 30a includes a body portion 34a that is essentially circular at the cross section, a neck portion 32a, and a mouth portion 33a that is essentially shaped so that it has a posterior portion that is longer than the anterior portion. In one embodiment, the mouth portion 33a is essentially triangular, ellipse, lens, or crescent in cross section wherein the narrower parts are in the posterior direction. In a preferred embodiment, the mouth portion 33a is essentially triangular in cross section.

In one embodiment, the urine collection device of the present invention is a shape that is suitable for a subject with male presenting genitalia. In this aspect of the invention, the urinal 30b includes a body portion 34b that is essentially square at the cross section, a neck portion 32a, and a mouth portion 33a that is essentially circular at the cross section.

It is another object of this invention to provide various features and/or attachment accessories that increase the comfort of the subject and the efficacy of collecting urine. Features and/or attachment accessories can include but are not limited to a handle member, cap member, drain member, valve member, and mouthpiece member.

Therefore, it is another object of this invention to provide a urine collection device further comprising a handle member 36 is attached or attachable to the anterior side.

It is also an object of the present invention to provide a urine collection device further comprising a cap member that covers mouth portion 33 and prevents urine from spilling out is attached or attachable to the anterior side.

It is a further object of this invention to provide a mouthpiece attachment member that is suitable to make the mouth portion 33 of a urinal a more comfortable to fit against a subject's body and/or increase the efficiency of urine collection. In one embodiment, the mouthpiece attachment member can be fitted onto mouth portion 33 like a cap but comprises an opening in its center that allows for urine to be collected in a urinal 30. The mouthpiece attachment member may comprise any material sufficient to achieve the objectives of the present invention, including but not limited to cushion, pad, foam, and/or cloth.

It is another object of this invention to provide a urine collection device with a drainage feature comprising a drain member 38a that is attached or attachable to the urinal 30 of the present invention and is means for draining the present urinal or removing the urine collected herein. The drain member 38a is shaped in a manner that allows it to be fit into drainage tubing as shown in FIGS. 5a and 5b. Drain member 38a is located on the urine collection device in any location suitable to efficiently drain the urine, for example on the posterior portion or the bottom portion.

In one embodiment, the present invention further comprises a valve member 38b on the drain member 38a, the valve member 38b being a means for controlling the flow of urine out of the urine collection device. Because, it is an object of the present invention to provide a stabilized urinal device that allows for the comfortable and efficient collection of urine from a subject wherein the urine can be drained from the urine collection device, and collected into another container, such as a drainage bag, for viewing, preferably, valve member 38b may be a means for controlling the flow of urine out of the urinal into the drainage tubing and a drainage bag.

It is an additional object of the present invention to provide an assembly that has antimicrobial properties by being made, coated, or washed with microbial inhibiting materials.

In another embodiment, the urine collection device is an external catheter and may utilize any existing condom catheter on the market. The urine collection assembly may also utilize an improved condom catheter described below.

The improved condom catheter is a means for collecting urine from a subject wherein the condom catheter is an overall shape that allows urine to be collected comfortably, and efficiently (less splash-back and spillage) from a subject while sitting down or laying down horizontally. The condom catheter can be formed as a unitary structure by molding, casting, machining, or by any known method to create the desired structure. The condom catheter of the present invention can be made of any type of suitable material, preferably any suitable plastic, including, latex.

In one embodiment, the condom catheter 30 is hollow inside and includes a body portion 34 that collects urine, a bottom portion 35, a neck portion 32, a head portion 31, and a mouth portion 33 that is an opening to collect urine, where the body portion 34 is a shape that is any shape suitable to collect urine, preferably essentially square cross section, the bottom portion is a shape that is suitable to collect urine, the neck portion 32 extends from the body portion 34 and is a shape that is suitable to contain urine, and the mouth portion 33 is a shape that allows to be fitted to a subject's body and collect urine comfortably.

It is another object of this invention to provide the condom catheter 30 of the present invention with a drainage feature comprising a drain member 38a that is attached or attachable to the urinal 30 of the present invention and is means for draining the present urinal or removing the urine collected herein. The drain member 38a is shaped in a manner that allows it to be fit into drainage tubing as shown in FIG. 10b. Drain member 38a is located on the condom catheter 30 in any location suitable to efficiently drain the urine, for example on the posterior portion or the bottom portion.

In one embodiment, the present invention further comprises a valve member 38b on the drain member 38a, the valve member 38b being a means for controlling the flow of urine out of the condom catheter 30. Because, it is an object of the present invention to provide a stabilized condom catheter 30 that allows for the comfortable and efficient collection of urine from a subject wherein the urine can be drained from the urine collection device, and collected into another container, such as a drainage bag, for viewing, preferably, valve member 38b may be a means for controlling the flow of urine out of the urinal into the drainage tubing and a drainage bag.

The present invention also relates to a urine collection assembly that allows a urine collection device to be attached, secured, held and/or stabilized against a subject's body. More specifically, the urine collection assembly comprises: i) the apparatus 1 as described above; and ii) a urine collection device as described above.

Another aspect of the present invention also includes a method of using the described apparatus to attach, hold, stabilize and/or secure a medical device in relation to a subject. The method comprises the steps of: i) attaching the apparatus to the medical device to form the assembly; ii) attaching the assembly comprising the apparatus and medical device to a subject's body; iii) adjusting the assembly as necessary; and iv) any sequential combination of i, ii, and iii thereof. In a preferred embodiment, the medical device is a urine collection device; in a more preferred embodiment the urine collection device is an external catheter or a portable urinal.

It is a further aspect of the present invention to provide a method of using the described assembly to attach, hold, secure and/or stabilize a portable urinal to a subject. The method comprises the steps of: i) attaching the apparatus to a urine collection device to form an assembly; ii) attaching the assembly to a subject's body; iii) adjusting the assembly if necessary to secure the urinal to the subject's body; and iv) optionally attaching or utilizing an attached accessory that increases the comfort or efficacy of collecting urine. In one embodiment, the portable urinal is the improved urine collection device comprising a drainage member as described herein. In a further embodiment, the method further comprises the steps of attaching a drainage tube to the improved urine collection device described herein and collecting the urine collected therein.

Another embodiment of the present invention is a kit comprising: i) a container comprising the apparatus or the apparatus and a urine collection device; and ii) an instruction set describing the use of the apparatus and or assembly comprising the apparatus and urine collection device.

In another embodiment of the present invention, the kits described herein may further comprise various attachment accessories that increase the comfort, convenience, or efficacy of the stabilization of the medical device against the body of a patient, including, but not limited to, a handle, cap, drain member, valve, and a mouthpiece attachment.

The invention illustratively described herein suitably may be practiced in the absence of any element or elements, limitation or limitations which is not specifically disclosed herein. Thus, for example, in each instance herein any of the terms “comprising,” “consisting essentially of and “consisting of may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An apparatus comprising:
a. a collar for attaching the apparatus to the outer surface of a medical device wherein the collar is adjustable collar; and
b. at least one strap for attaching the medical device against the body of a subject wherein the at least one strap is adjustable.

2. The apparatus according to claim 1 comprising at least one, two, three, or four adjustable straps for attaching the medical device against the body of a subject.

3. The apparatus according to claim 1 wherein the collar and at least one strap comprise a separable fastener or non-separably fastener.

4. The apparatus according to claim 1 wherein the medical device is a urine collection device.

5. A urine collection assembly comprising (i) an apparatus and (ii) a urine collection device wherein the apparatus comprises:
a. a collar for attaching the apparatus to the urine collection device; and
b. at least one adjustable strap for attaching the assembly to the body of a subject.

6. The urine collection assembly according to claim 5 comprising at least one, two, three, or four adjustable straps.

7. The urine collection assembly according to claim 5 wherein the urine collection device is a portable male or female urinal or an external catheter.

8. A method of stabilizing a medical device comprising the steps of:
a. attaching the apparatus according to claim 1 to a medical device to form an assembly; and
b. attaching the assembly to the body of a subject.

9. The method according to claim 8 wherein the medical device is a urine collection device.

10. The method according to claim 9 wherein the urine collection device is a portable male or female urinal or an external catheter.

11. A kit comprising a container comprising the apparatus according to claim 1 and an instruction set describing the use of the apparatus.

12. The kit of claim 11 further comprising a urine collection device.

13. The kit of claim 12 wherein the urine collection device is a portable male or female urinal or an external catheter.

14. The kit of claim 13 wherein the kit optionally further comprises attachment accessories comprising a handle, cap, drain, valve, and a mouthpiece attachment.
